SEQUENCE LISTING (28 sequences)

SEQ ID NO: 1 - protein, 275 aa, Corynebacterium glutamicum 0807
MSTALPDQLKWEYSAFPVQISQKQRLSPGFMRITVTGDKLRFFGQWGLDQRIKLIIPSPAGNIPDFGILDEPTPPPTTWLPRAKSFPADQRPILRTYTPSAVRPELCEVDIDIYLHNPSGPVSRWAKNCSVDDELIITGPDVRAGETGYGITYHPTSAIDRLCLIGDCASAPAIANIVNQSKVPTTVFLHVDSLEDDVLIADSSTKLTFEDIDAYKAKVFQWASANAADPSVHFWIAGETSMVRFIRKELINSYRVDSSRITFLGYWKYGRRTVD

SEQ ID NO: 2 - DNA, 828 bp, Corynebacterium glutamicum 0807
atgtccacagctctccccgatcagctcaagtgggaatacagtgccttccccgtgcagatctcgcagaagcaacggcttagtcccggcttcatgcggatcaccgtcactggtgacaagctccgattctttggccagtggggtttggaccaacgcatcaaactgatcattccaagcccggctgggaacatcccagatttcggaattctcgacgaacccactcccccaccgacaacgtggcttcctcgtgctaagtcttttccagcggaccaacgaccgatcttgcgcacctacaccccatctgcggtccgacccgaactatgcgaagtagacattgatatctatcttcacaacccttcgggaccagtatccagatgggcaaagaactgcagtgttgacgatgaactaatcatcaccggccctgacgtacgcgcaggagaaaccggctacggaatcacctatcatccgacttctgcgatcgatcgcctctgtctcatcggcgattgtgcatcagctcccgcgatcgcaaatatcgtcaatcaatcaaaagtacctactacggttttcctccacgtagacagcctagaagatgatgtattgatcgccgatagctccaccaagctcactttcgaagacatcgacgcttacaaagcaaaggtcttccaatgggcttcagccaatgcagcagatccttcagtacacttctggatcgccggtgaaactagcatggtgcgcttcattcgcaaagaactaatcaacagctaccgagttgattcctcacgaatcactttcctcggctactggaaatacggccgacgaaccgtagactag

SEQ ID NO: 3 - protein, 188 aa, Corynebacterium glutamicum 0600
MADTERELADLVPQATAGDRRALQRIMEIIHPIVLRYARARIGGGRQPTAEDVAQEICLAVATSIRNFVDQGRPFMAFVYGIASNKVADAHRAMSRDKSTPIEEVPETSPDTFTPEDFALVSDGSNRVRELLDLLSEKARDILILRVIVGLSAEETAEMVGSTPGAVRVAQHRALTTLRSTLEQQENK

SEQ ID NO: 4 - DNA, 567 bp, Corynebacterium glutamicum 0600
ttggctgatactgagcgcgagctcgctgacctggtaccgcaggcaacggcgggcgatcgtcgggcattgcaaagaataatggagattattcaccccattgttttgcgttatgctcgcgctcgtattggaggtggacgccagccaacggcagaagacgttgctcaagaaatctgcctggcggtagccacctccattaggaactttgtcgaccagggtaggccgttcatggcgtttgtctacggcattgcatctaacaaggtcgcagatgctcacagggcgatgtcgagggataaatcgactcctattgaggaagtcccagaaacttcaccagatacttttacccccgaagactttgcgctggtcagcgatggaagtaacagagttagggaacttctcgatctactgagtgaaaaggcacgcgacattcttatcttgagagttatcgttggtctttccgcagaagaaactgcagagatggtgggcagcaccccaggtgctgtacgagttgcccaacacagggcactcacgacacttcgaagcacacttgagcagcaggagaacaagtaa

SEQ ID NO: 5 - protein, 230 aa, Corynebacterium glutamicum 1427
MPAGGLIVAIDGPSGTGKSTTSRALATRLSAKYLDTGAMYRVATLHVLNQGIDPADSAAVIAATAVLPLSISDDPASTEVLLAGVDVQKDIRGPEVTQNVSAVSAIPEVRENLVALQRALAAKAHRCVVEGRDIGTAVLVDAPIKAFLTASAEVRAQRRFDQDTAAGRDVDFDAVLADVVRRDELDSTRAASPLKPADDAHIVDTSDMTMDQVLDHLIHLVEASAERSNQ

SEQ ID NO: 6 - DNA, 693 bp, Corynebacterium glutamicum 1427
atgcctgccggtggcctcatcgtagccatcgacgggccgtctggcaccggaaaatccaccacatcccgcgcgctcgcaacccgtctctcggccaagtacctagatactggtgcgatgtaccgcgtcgcaacgcttcatgtgcttaaccaggggattgaccctgcagatagcgcagccgtgatcgctgcaaccgctgtattgccgttgtcgatttctgacgatcccgcctccactgaggtgttgctcgcgggcgtcgatgtgcaaaaggacatccgcggaccagaagtcacccaaaatgtctccgcagtgtccgcgatccctgaggttcgtgaaaacttggtggcgttgcagcgcgcactcgccgccaaagcacatcgctgcgtcgtcgaaggcagagacatcggaacggcagtgcttgtcgacgcgcccatcaaggcgtttctcaccgcctcagcggaagtccgcgcccagcgacgctttgaccaagacaccgcagcaggtcgcgacgtagatttcgacgctgtgctggcagatgttgttcgccgcgatgaactagattccacccgtgccgcctcaccgctgaaaccagcagatgatgcacacatcgtggacacctctgatatgaccatggatcaagtacttgatcacctcatccacctagtggaagcctccgctgaaaggagcaaccagtga

SEQ ID NO: 7 - protein, 105 aa, Corynebacterium glutamicum 1102
MTYTIAQPCVDVLDRACVEECPVDCIYEGKRMLYIHPDECVDCGACEPACPVEAIFYEDDVPDEWLDYNDANAAFFDDLGSPGGAAKLGPQDFDHPMIAALPPQA

SEQ ID NO: 8 - DNA, 318 bp, Corynebacterium glutamicum 1102
atgacatacacaatcgcacagccctgcgttgacgtcttggatcgtgcctgcgttgaagaatgcccagtagattgcatctacgaaggtaagcgcatgctgtacatccacccggatgagtgcgttgactgtggtgcatgtgagcctgcttgcccagttgaggcaatcttctacgaggacgatgtcccagacgaatggcttgactacaacgatgccaacgctgcattcttcgatgatctgggctccccaggtggtgcggctaagcttggaccacaagattttgatcacccaatgatcgctgcgctgccgcctcaggcataa

SEQ ID NO: 9 - protein, 474 aa, Corynebacterium glutamicum 2859
MTPTRRILLWAWTTVLLGSLLWPLAAPGELLLRDMSVVDHPALSLNALGFGDLPSRNAPQDGVLALLGFLPVSWIVRTMLLVAGFAGAWGAMRLGPSKFLAVTVAIYNPFVVERLLQGHWSLVMAVWLLPLVVALRRHPRWQVVAIWAASLTPTGAVVAAVTGVASSKRKRFTTLCSFLSWLPWLIPALLATPTSGGALTFAIRSETYAGTLGTALGLGGIWNAGAVPASRELGFAVAGILLFAILLAGFKNCPWVLALLAVVGFMGAIGPWLMPNLFTWTIAYVPGAALFRDSQKLLMLAIPAYVCLAAGVKSPLSWVATGLALLQIPDAPREVSVIRPSSAHVESVEALAEIADGRDILIIGQGPLVTREDGIPVVDPKTKALSVVESGELRVDGIITDAPSQRWTEATQAWAAGDIERLEELGVGVVVDGDTITETGAPPQHGWKYYLGVGLTVLWMALPLGLLFRRKTKK

SEQ ID NO: 10 - DNA, 1425 bp, Corynebacterium glutamicum 2859
atgaccccgacccgccgtatcctgctgtgggcatggacgactgtgcttttgggttctttgctgtggccgttggctgcgcctggcgagttgttgttgcgggatatgtcggtggtggaccatcctgcgttgtcgttgaatgcgttgggttttggtgatttgccgtcgcggaacgccccgcaggatggtgtgctggcgttgcttggctttttgccggtgagttggatcgttcgaaccatgctgcttgtggcgggtttcgcgggcgcgtggggtgcgatgcgccttgggccttccaagtttttggccgttaccgtggcgatttacaatcccttcgtggtggagcgtctgctgcagggccattggtcgttggtgatggcggtgtggctgttgccgctggttgtggcgctgcgcaggcatccgcgttggcaggttgtggcgatctgggcggcgtcgttgacgcccacgggtgcggtggttgcggcggtcacgggcgtggcgagttctaaaagaaaacgctttacgacgctatgttccttcctttcgtggcttccttggctaatacctgcgcttcttgccacccccacttcgggaggtgcgctgaccttcgccattcgttctgaaacatatgcaggaacgttgggaactgcgctgggcctgggtggaatttggaacgcgggggccgtgccggcctcacgcgaactgggtttcgcggttgctggaattttgttatttgcgattctgctggcgggtttcaaaaactgtccgtgggttctcgcactgctggcggtcgtaggttttatgggggcgatcggtccgtggctgatgccgaatctgttcacgtggaccatcgcatatgttccaggcgccgcgctgtttagggattctcaaaaactcctcatgctggctatccctgcctatgtgtgtttggccgccggggtgaaaagcccactgtcgtgggtggctaccggtttggcgttgctccagattcctgatgcaccacgtgaggtttccgtgatacgcccaagttcagcgcatgtggaatcagtggaagcactggcagaaatcgctgatggccgcgacatcttaatcatcggccaaggccccttggtgacccgcgaggatgggatcccggttgtcgatcccaaaaccaaagccctctccgtggtggaatccggcgaactgcgtgtggacggaatcatcaccgacgcgccctcacagcggtggaccgaagcaacgcaggcatgggcggccggggatatcgagcgccttgaagaacttggcgttggtgtcgtggtggatggagatacgatcacagaaactggcgcaccaccgcagcatggctggaaatactacctcggtgtgggcctgaccgtgctgtggatggcgttgccgctgggactactttttcgacgcaagaccaagaagtag

SEQ ID NO: 11 - protein, 301 aa, Corynebacterium glutamicum 2857
MNPRWRMGAYDWVDIISTCEFSGKVWAVFMKRSATVLIIAGVLFLIFAFTVPPYVTGQARTIPKDLDLTLVSESPQGFVRTEHIVTAPTEKVDEIATHVDQTVTDVQGKTVAEISDDVVLIGHSRYPVIKPTATISGSPADSSNVVREGLHYFFPANTLRNSYPYYDIVLGEDSPVDYVSREGNTYTFYQHLRYVPLDDSHTYSVERTLKVDRFSGIIVAKDEAMTFHGPDGDDTVEFTYTADTLKLLQDHAHDIDQRLSWAKGFDFFSKFLGLSLLAIGVFLTGIFKRGQLMSTVNKLRS

SEQ ID NO: 12 - DNA, 906 bp, Corynebacterium glutamicum 2857
atgaatccgcgatggcggatgggtgcatatgattgggtagacattatttcaacatgcgagtttagcggaaaggtgtgggctgtttttatgaagcgatctgcaacggtcctcattattgcgggcgtgctgttcctcatttttgccttcacggtaccgccgtatgtgactggtcaggcgcggacgattccgaaggatttggatctgacgttggtgagcgaaagtccgcaggggtttgtgcgcactgaacatattgtgactgctccgacggaaaaggtcgatgagatcgcgacgcatgtggatcagacagttacggatgtgcaggggaaaactgttgcggaaatttcggatgatgtggtgttgattggacactctcgttatccggtgattaagccgactgccaccatttcgggttcgccggcggatagtagcaatgtggtgcgggaggggttgcattacttcttcccggctaatacgttgcggaattcttatccctattatgacatcgtattgggtgaggattccccggtggattatgtctcgcgcgagggcaatacttataccttctaccagcatcttcgttatgttccattggatgattctcacacctattcggtggagcggaccctgaaagtggatcgtttttccggcatcattgtggctaaagatgaggcgatgacgtttcatggcccagacggcgatgacacagtagaattcacttatactgcggatacgttgaagcttctgcaggatcatgcgcatgatattgatcagcggttgtcgtgggctaaggggtttgatttcttttctaaattcttaggcctgtcgttgcttgcgattggtgtgttcctcacgggaattttcaagcgcggccagctgatgagcactgtgaataaactcaggagttaa

SEQ ID NO: 13 - protein, 334 aa, Corynebacterium glutamicum 2525
MAADSDLSVHDAYLKEHVAPVKAINWNSIPDSKDLEVWDRLTGNFWLPEKVPVSNDIKSWGTLNEVEKAATMRVFTGLTLLDTIQGTVGAISLLPDADSLHEEAVLTNIAFMESVHAKSYSNIFMTLASTAEINDAFRWSEENENLQRKAKIILSYYEGDDPLKRKIASVILESFLFYSGFYLPMYWSSHSKLTNTADVIRLIIRDEAVHGYYIGYKYQKAVAKETPERQEELKEYTFDLLYDLYDNETQYSEDLYDDLGWTEDVKRFLRYNANKALNNLGYEGLFPADETKVSPNILSALSPNADENHDFFSGSGSSYVIGKAENTEDDDWDF

SEQ ID NO: 14 - DNA, 1005 bp, Corynebacterium glutamicum 2525
atggctgctgattctgatctcagtgttcacgatgcttacttaaaggagcatgttgcacctgtaaaggcgatcaactggaactccatcccagattccaaagatcttgaagtctgggatcgtctgaccggtaacttctggctcccagaaaaggtcccagtatccaacgacatcaagagctggggaaccctcaacgaggttgaaaaagccgcaaccatgcgcgtgttcaccggacttaccctgctggacaccattcagggcactgtcggcgcaatctccctgcttccagacgcagattcactgcacgaagaagcggtgctaaccaacattgcgttcatggaatccgtgcacgcaaagagttactccaacatcttcatgactctggcctccaccgcggaaatcaacgatgcgttccgttggtctgaggaaaatgaaaacctgcagcgcaaggcaaagatcatcctgtcttactatgagggcgatgatccactaaagcgcaagatcgcctccgtgatcctggagtccttcctgttctactccggcttctacctcccaatgtattggtccagccactccaagctgaccaacaccgccgacgtgatccgcctgatcatccgcgatgaggcagtgcacggctactacattggctacaagtatcaaaaggctgtcgcgaaggagactccagagcgtcaggaagagctgaaggagtacaccttcgatctgctctacgatctttacgataacgaaactcagtactccgaagatctctacgacgatcttggatggaccgaggatgttaagcgattccttcgctacaacgccaacaaggccctcaacaaccttggctacgaaggactcttcccagcggatgaaaccaaggtgtccccaaacatcttgtctgcgctgtcaccaaacgctgatgagaaccacgacttcttctccggctccggttcctcttacgttattggtaaggcagaaaacaccgaggatgatgactgggacttctaa

SEQ ID NO: 15 - protein, 707 aa, Corynebacterium glutamicum 2530
MPRGDQMDFHALNALLNLYDDNGKIQFEKDREAANQYFLQHVNQNTVFFHNLQEKIDYLVENKYYDPIVLDKYDFQFIKDLFKRAYGFKFRFQSFLGAYKYYTSYTLKTFDGRRYLERFEDRVCMVALTLADGDRALAENLVDEIMSGRFQPATPTFLNSGKAQRGEPVSCFLLRIEDNMESIGRSINSALQLSKRGGGVALLLSNLREAGAPIKKIENQSSGVIPVMKLLEDAFSYANQLGARQGAGAVYLNAHHPDILSFLDTKRENADEKIRIKTLSLGVVIPDITFELAKRNDDMYLFSPYDVERIYGKPFADVSITEHYDEMVDDDRIRKTKINARQFFQTLAEIQFESGYPYIMYEDTVNASNPIEGRITHSNLCSEILQVSTPSEFNDDLTYAEVGEDISCNLGSLNVAMAMDSPNFEKTIETAIRGLTAVSEQTSIDSVPSIRKGNEAAHAIGLGQMNLHGYFGREHMHYGSEEALDFTNAYFAAVLYQCLRASNKIATERGERFKNFENSKYATGEYFDDFDANDFAPKSDKVKELFAKSNIHTPTVEDWAALKADVMEHGLFNRNLQAVPPTGSISYINNSTSSIHPIASKIEIRKEGKIGRVYYPAPHMDNDNLEYFEDAYEIGYEKIIDTYAVATKYVDQGLSLTLFFKDTATTRDINRAQIYAWRKGIKTLYYIRLRQVALEGTEVDGCVSCML

SEQ ID NO: 16 - DNA, 2124 bp, Corynebacterium glutamicum 2530
gtgccacgcggagaccagatggacttccacgctcttaacgcgttgctcaacctttacgatgacaacggcaagatccagtttgagaaagaccgtgaagctgcaaaccagtacttcctgcagcacgtcaaccagaacaccgtcttcttccacaacctgcaggaaaagatcgactacctggttgaaaacaagtactatgacccaatcgttctggacaagtacgacttccagttcatcaaggacctcttcaagcgcgcatacggattcaagttccgcttccagtccttcctcggtgcatacaagtactacacttcctacaccctgaagaccttcgacggtcgccgctacctcgagcgtttcgaagaccgtgtctgcatggtcgccctcaccctcgctgacggcgaccgcgcattggccgagaacctggtcgatgagatcatgtctggccgtttccaaccagcaaccccaaccttcctgaactccggcaaggcacagcgcggcgagccagtatcctgcttcctcctgcgtatcgaagacaacatggagtccatcggacgttccatcaactctgctcttcagctgtccaagcgtggcggtggcgtagcgttgctgctgtccaaccttcgtgaagccggtgcaccgattaagaagattgaaaaccagtcttccggtgttatcccagtgatgaaacttctggaagatgctttctcctacgctaaccagctgggtgctcgtcagggtgcaggtgctgtgtacctcaacgctcaccacccagatatcctgtccttcctggataccaagcgtgagaacgccgatgagaagatccgcatcaagaccctgtccctgggtgttgtgattccggacatcaccttcgagctggctaagcgcaacgatgacatgtacctgttctccccatacgatgtggagcgcatttacggcaagcctttcgcagacgtctcaatcaccgagcactacgacgagatggtggatgatgaccgcatccgcaagaccaagatcaacgcgcgtcagttcttccagaccctggcagaaatccagttcgagtccggttacccatacatcatgtatgaagacaccgtgaatgcatccaacccaatcgaaggtcgcatcacccactcaaacctgtgctctgagatccttcaggtgtccaccccatctgaattcaacgatgacctgacttacgcagaggtcggcgaagacatttcttgtaacttgggttccctcaacgttgcaatggctatggattcaccaaactttgagaagaccatcgaaaccgcaatccgcggcttaactgcagtgtctgagcagaccagcatcgattccgtgccttccatccgtaagggcaacgaagcagctcacgccatcggccttggccagatgaaccttcacggctacttcggtcgcgagcacatgcactacggctccgaggaagccctggacttcaccaacgcatactttgctgccgtgctgtaccagtgcctgcgtgcatccaacaagatcgctactgagcgtggagagcgtttcaagaacttcgaaaactccaagtatgcaaccggtgagtacttcgatgatttcgatgcaaacgacttcgcaccaaagtccgacaaggtcaaggaactctttgccaagtcgaacatccacaccccaaccgttgaggactgggctgcgctgaaggccgacgtgatggagcacggtctgttcaaccgtaacctgcaagcggttccaccaaccggttcgatctcctacatcaacaactccacctcgtcgatccacccaatcgcatccaagattgagatccgcaaggaaggcaagatcggccgcgtttactacccagctccacacatggacaatgacaaccttgagtacttcgaggacgcctacgaaatcggctacgagaagatcattgacacctacgctgtggcaaccaagtacgttgaccagggcctgtcactgaccttgttcttcaaggacactgccaccacccgtgacatcaaccgtgcgcagatctacgcatggcgcaagggcatcaagaccttgtactacattcgcctgcgccaggttgctctggaaggcactgaagttgacggctgcgtcagctgcatgctgtaa

SEQ ID NO: 17 - protein, 148 aa, Corynebacterium glutamicum 2531
MLIVYFSSATDNTHRFVQKLDLPNVRIPLTRVEEPLKINEPYVLITPTYGGGVSMTGENSRPVPPQVIRFLNDEHNRSFIRAVVAGGNSNFGSDFGLAGEIISKKCKVPYVYRFELMGNEEDVSILRGGLTQNAQALGLEPQEPVTSR

SEQ ID NO: 18 - DNA, 447 bp, Corynebacterium glutamicum 2531
atgctaatcgtgtatttttcctcggccaccgacaacacgcatcgttttgtacaaaagctcgatttacccaacgtgcgcatccccctcactagggtggaagaaccgctgaaaatcaacgagccctacgtgctaatcaccccgacctatggtggtggagtctccatgactggagaaaactcccgcccggtcccaccacaagtcatcaggtttttaaatgatgaacacaaccgcagcttcatcagggcagttgttgcaggtggaaactcaaacttcggctccgattttgggttggcaggcgagatcatttccaagaaatgtaaagtgccctatgtctaccgtttcgagctcatgggcaatgaggaagatgtaagtatccttcgtggaggtcttactcaaaacgcccaagctttggggctggaaccacaagaaccagttacctcgcgataa

SEQ ID NO: 19 - protein, 77 aa, Corynebacterium glutamicum 2532
MAITVYTKPACVQCNATKKALDRAGLEYDLVDISLDEEAREYVLALGYLQAPVVVADGSHWSGFRPERIREMATAAA

SEQ ID NO: 20 - DNA, 234 bp, Corynebacterium glutamicum 2532
atggcaatcaccgtttacaccaagccagcttgcgtccagtgcaatgccaccaagaaggccctcgaccgcgctggtcttgagtatgacctcgttgatatcagccttgatgaagaggcacgtgagtacgtcctcgcacttggctacctgcaggcaccagttgtcgttgcagatggctcccactggtccggtttccgcccagagcgcatccgtgaaatggcaaccgcagctgcctaa

SEQ ID NO: 21 - DNA, 26 bp, Artificial Sequence
  Primer
ggtggatccggtggaatttggcctgc

SEQ ID NO: 22 - DNA, 23 bp, Artificial Sequence
  Primer
tcaggatcccatccgggtgagac

SEQ ID NO: 23 - DNA, 20 bp, Artificial Sequence
  Primer
gtaggatcccactggttgcc

SEQ ID NO: 24 - DNA, 25 bp, Artificial Sequence
  Primer
gaaggatccaactcaccggcaaaaa

SEQ ID NO: 25 - DNA, 26 bp, Artificial Sequence
  Primer
ctgggtaccggagtgttttgggttgt

SEQ ID NO: 26 - DNA, 26 bp, Artificial Sequence
  Primer
cacggtaccagcggaattcgcggaaa

SEQ ID NO: 27 - DNA, 27 bp, Artificial Sequence
  Primer
atgggatcctagtggcgataatttagg

SEQ ID NO: 28 - DNA, 26 bp, Artificial Sequence
  Primer
cttggatccaaaggtgtgaaggggtt

(19) United States
(12) Patent Application Publication
(10) Pub. No.: US 2012/0034669 A1
(43) Pub. Date: Feb. 9, 2012

(54) PROCESS FOR PRODUCING USEFUL SUBSTANCE

(75) Inventors: Masato Ikeda, Kamiina-gun (JP); Seiki Takeno, Kamiina-gun (JP); Satoshi Mitsuhashi, Tsukuba-shi (JP)

(73) Assignees: KYOWA HAKKO BIO CO., LTD., Tokyo (JP); SHINSHU UNIVERSITY, Matsumoto-shi (JP)

(21) Appl. No.: 13/201,806

(22) PCT Filed: Feb. 17, 2010
(86) PCT No.: PCT/JP2010/052341

§ 371 Date: Aug. 16, 2011

(30) Foreign Application Priority Data
Feb. 18, 2009 (JP) 2009-034692

(51) Int. Cl. C12P 1/04

(52) U.S. Cl. 435/170

(57) ABSTRACT

The present invention provides a process for producing a useful substance which comprises culturing a transformant obtained by transforming a microorganism capable of producing the useful substance with a DNA encoding a protein in a medium, wherein the protein has an activity to improve growth of a high concentration oxygen-requiring microorganism under low oxygen concentration; forming and accumulating the useful substance in a culture; and collecting the useful substance from the culture.

### TECHNICAL FIELD

The present invention relates to a process for producing a useful substance using a microorganism transformed with a DNA encoding a protein that has an activity to improve the growth of a high concentration oxygen-requiring microorganism under low oxygen concentration.

### BACKGROUND ART

Aerobic fermentation of a useful substance such as an amino acid, a nucleotide, etc., requires a large amount of oxygen. Insufficient oxygen causes a problem such that a toxic organic acid is accumulated in place of a desired product. For this reason, it is important to keep dissolved oxygen in a medium during culturing at more than a certain level. To overcome this problem, fermentation tanks having a high oxygen supply efficiency or oxygen enrichment membranes have been developed; however, the installation of such devices requires high costs. Therefore, a microorganism that does not require, during culturing, a large amount of dissolved oxygen is demanded.

Various genes that are assumed to be involved in oxygen utilization or oxygen stress resistance have been found on the genome of Corynebacterium glutamicum. For example, genes involved in a respiratory chain has been identified (see NPL 1). The sigB gene (see NPL 2) or sigD gene (see NPL 1), which are the sigma factors of RNA polymerase, and the hemoglobin-like glbO gene (see PTL 2) have been found. Further, a group of genes (nrdHIE and nrdF) involved in ribonucleotide reductase has been identified (see NPL 3). Information of these genes are disclosed in the publication of a patent application (see PTL 3).

However, effects of enhanced expression of these genes on the dissolved oxygen level that a microorganism requires or on the amino acids productivity have not been known.

### CITATION LIST

### Patent Literature

PTL 1: U.S. Pat. No. 6,890,744

PTL 2: U.S. Pat. No. 6,759,218

PTL 3: Japanese Unexamined Patent Publication No. 2002-191370 Non-patent Literature

NPL 1: J. Biotechnol., 104, 129 (2003)

NPL 2: BMC Genomics, 8, 4 (2007)

NPL 3: Microbiology, 145, 1595 (1999)

### SUMMARY OF INVENTION

### Technical Problem

The present invention relates to a process for producing a useful substance using a microorganism transformed with a recombinant DNA that contains a DNA encoding a protein that has an activity to complement the high concentration oxygen-requiring properties of microorganism.

### Solution to Problem

The present invention provides the following items (1) to (3).

(1) A process for producing a useful substance which comprises culturing a transformant obtained by transforming a microorganism capable of producing the useful substance with a DNA encoding a protein in a medium, wherein the protein has an activity to improve growth of a high concentration oxygen-requiring microorganism under low oxygen concentration; forming and accumulating the useful substance in a culture; and collecting the useful substance from the culture.
(2) The process according to Item (1), wherein the protein having the activity to improve the growth of the high concentration oxygen-requiring microorganism under low oxygen concentration is a protein defined in any one of [1] to [6] below:

[1] a protein comprising the amino acid sequence shown in SEQ ID NO: 1, 3, 5, 7, 9 or 11;

[2] a protein consisting of the amino acid sequence, wherein one or more amino acid residues are deleted, substituted or added in the amino acid sequence shown in SEQ ID NO: 1, 3, 5, 7, 9 or 11, and having the activity to improve the growth of the high concentration oxygen-requiring microorganism under low oxygen concentration;

[3] a protein consisting of the amino acid sequence which has 80% or more homology or identity to the amino acid sequence shown in SEQ ID NO: 1, 3, 5, 7, 9 or 11, and having the activity to improve the growth of the high concentration oxygen-requiring microorganism under low oxygen concentration;

[4] a protein consisting of the amino acid sequence shown in SEQ ID NO: 13, 15, 17 or 19;

[5] a protein consisting of the amino acid sequence, wherein one or more amino acid residues are deleted, substituted or added in the amino acid sequence shown in SEQ ID NO: 13, 15, 17 or 19, and having the activity to improve the growth of the high concentration oxygen-requiring microorganism under low oxygen concentration; and

[6] a protein consisting of the amino acid sequence which has 80% or more homology or identity to the amino acid sequence shown in SEQ ID NO: 13, 15, 17 or 19, and that having the activity to improve the growth of the high concentration oxygen-requiring microorganism under low oxygen concentration. (3) The process according to Item (1) or (2), wherein the DNA encoding the protein having the activity to improve the growth of the high concentration oxygen-requiring microorganism under low oxygen concentration is a DNA defined in any one of [1] to [6] below:

[1] DNA encoding the protein consisting of the amino acid sequence shown in SEQ ID NO: 1, 3, 5, 7, 9 or 11; [2] DNA comprising the nucleotide sequence shown in SEQ ID NO: 2, 4, 6, 8, 10 or 12;

[3] DNA which hybridizes with a DNA consisting of the nucleotide sequence complementary to the nucleotide sequence shown in SEQ ID NO: 2, 4, 6, 8, 10 or 12 under stringent conditions, and codes for the protein having the activity to improve the growth of the high concentration oxygen-requiring microorganism under low oxygen concentration;

[4] DNA encoding the protein consisting of the amino acid sequence shown in SEQ ID NO: 13, 15, 17 or 19;

[5] DNA consisting of the nucleotide sequence shown in SEQ ID NO: 14, 16, 18 or 20; and

[6] DNA which hybridizes with DNA consisting of the nucleotide sequence complementary to the nucleotide sequence shown in SEQ ID NO: 14, 16, 18 or 20 under stringent conditions, and encodes for the protein having the activity to improve the growth of the high concentration oxygen-requiring microorganism under low oxygen concentration.

### Advantageous Effects of Invention

According to the present invention, a process for producing a useful substance can be provided in which a transformant transformed with a DNA encoding a protein that has an activity to improve the growth of a high concentration oxygen-requiring microorganism under low oxygen concentration is used.

### DESCRIPTION OF EMBODIMENTS

Any microorganism can be used as the transformant used in the production process of the present invention, insofar as the microorganism is a transformant obtained by transforming a microorganism having an ability to produce a useful substance with a DNA encoding a protein that has an activity to improve the growth of a high concentration oxygen-requiring microorganism under low oxygen concentration.

Examples of such microorganisms include those belonging to the genera Corynebacterium, Brevibacterium, Microbacterium, Escherichia, Serratia, Bacillus, Pseudomonas and Streptomyces; preferably those belonging to the genera Corynebacterium, Brevibacterium and Microbacterium; and more preferably those belonging to the genus Corynebacterium.

Specific examples of microorganisms belonging to the genus Corynebacterium include Corynebacterium acetoacidophilum (e.g., Corynebacterium acetoacidophilum ATCC13870), Corynebacterium acetoglutamicum (e.g., Corynebacterium acetoacidophilum ATCC15806), Corynebacterium callunae (e.g., Corynebacterium callunae ATCC15991), Corynebacterium glutamicum (e.g., Corynebacterium glutamicum ATCC13032, ATCC13060, ATCC13826, ATCC14020, and ATCC13869), Corynebacterium herculis (e.g., Corynebacterium herculis ATCC13868), Corynebacterium lilium (e.g., Corynebacterium lilium ATCC15990), Corynebacterium melassecola (e.g., Corynebacterium melassecola ATCC17965), Corynebacterium thermoaminogenes (e.g., Corynebacterium thermoaminogenes ATCC9244) and the like.

Specific examples of microorganisms belonging to the genus Brevibacterium include Brevibacterium saccharolyticum (e.g., Brevibacterium saccharolyticum ATCC14066), Brevibacterium immariophilum (e.g., Brevibacterium immariophilum ATCC14068), Brevibacterium roseum (e.g., Brevibacterium roseum ATCC13825), Brevibacterium thiogenitalis (e.g., Brevibacterium thiogenitalis ATCC19240) and the like.

Specific examples of microorganisms belonging to the genus Microbacterium include Microbacterium ammoniaphilum (e.g., Microbacterium ammoniaphilum ATCC15354) and the like.

The useful substance produced in the present invention can be any substance that can be produced by a microorganism belonging to coryneform bacteria and that is considered to be industrially useful. Examples of the useful substance include amino acids; nucleic acids; vitamins; proteins such as various enzymes; peptides such as glutathione; sugars such as xylose; sugar alcohols such as xylitol; alcohols such as ethanol; organic acids such as lactic acid and succinic acid; lipids; and the like. Amino acids, nucleic acids, and vitamins are preferable; and amino acids are particularly preferable.

Examples of amino acids include L-alanine, glycine, L-glutamine, L-glutamic acid, L-asparagine, L-aspartic acid, L-lysine, L-methionine, L-threonine, L-leucine, L-valine, L-isoleucine, L-proline, L-histidine, L-arginine, L-tyrosine, L-tryptophan, L-phenylalanine, L-serine, L-cysteine, L-3-hydroxyproline, L-4-hydroxyproline and the like. Examples of nucleic acids include inosine, guanosine, inosinic acid, guanylic acid, and the like. Examples of vitamins include riboflavin, thiamine, ascorbic acid and the like.

The “high concentration oxygen-requiring microorganism” in the present invention means a microorganism that is incapable of growing under low oxygen concentration (from 0.5% to atmospheric condition (about 21%), e.g., 0.5%, 1%, 3%, 6%, 9 etc.) in which a wild-type microorganism can grow, or a microorganism that is capable of growing under such conditions; however, the growth speed thereof is reduced to 50% or less, preferably 20% or less, and more preferably 10% or less compared with that of the wild-type strain.

The aforementioned low oxygen concentration can be set using an oxygen absorbent and a special hermetic container that are sold for simple cultivation of anaerobic bacterium or microaerophilic bacterium. In this method, an oxygen absorbent (“Anaero Pack series” produced by Mitsubishi Gas Chemical Co., Inc.), a plate medium and an aversion indicator are put and cultured in the hermetic container. The amount of oxygen absorbent can be adjusted according to the manual of the manufacturer to thereby formulate several different low oxygen concentrations.

In a medium for determining whether the microorganism is a high concentration oxygen-requiring microorganism, saccharides such as glucose, fructose, sucrose, maltose and starch hydrolyzate, alcohols such as ethanol, organic acids such as acetic acid, lactic acid and succinic acid can be used as a carbon source.

Examples of nitrogen sources include various inorganic and organic ammonium salts such as ammonia, ammonium chloride, ammonium sulfate, ammonium carbonate and ammonium acetate, urea and other nitrogen-containing compounds, nitrogen-containing organic substances such as meat extract, yeast extract, corn steep liquor and soybean hydrolysate and the like.

Examples of inorganic salts include dipotassium hydrogen phosphate, potassium dihydrogen phosphate, ammonium sulfate, sodium chloride, magnesium sulfate, calcium carbonate and the like.

If necessary, trace nutrient sources such as biotin, thiamine, etc., can also be added. Such trace nutrient sources can be substituted with a meat extract, yeast extract, corn steep liquor, soybean hydrolysate, casamino acid and other medium additives.

Whether the microorganism is a high concentration oxygen-requiring microorganism can be confirmed by culturing the microorganism at 20 to 42° C., preferably 30 to 40° C. for 1 to 6 days under the aforementioned low oxygen concentration, and comparing the growth speed thereof with that of a wild-type strain.

Examples of the high concentration oxygen-requiring microorganism include Corynebacterium glutamicum strains OX-3, OX-96, OX-109, OX-112, OX-119 and OX-150.

In the present invention, the protein having an activity of improving the growth of a high concentration oxygen- requiring microorganism under low oxygen concentration indicates a protein that allows or improves the growth of the microorganism under low oxygen concentration when the high concentration oxygen-requiring microorganism is transformed by a DNA encoding the protein.

Examples of the protein include the following:

[1] a protein comprising the amino acid sequence shown in SEQ ID NO: 1, 3, 5, 7, 9 or 11;

[2] a protein consisting of the amino acid sequence, wherein one or more amino acid residues are deleted, substituted or added in the amino acid sequence shown in SEQ ID NO: 1, 3, 5, 7, 9 or 11, and having the activity to improve the growth of the high concentration oxygen-requiring microorganism under low oxygen concentration;

[3] a protein consisting of the amino acid sequence which has 80% or more homology or identity to the amino acid sequence shown in SEQ ID NO: 1, 3, 5, 7, 9 or 11, and having the activity to improve the growth of the high concentration oxygen-requiring microorganism under low oxygen concentration;

[4] a protein consisting of the amino acid sequence shown in SEQ ID NO: 13, 15, 17 or 19;

[5] a protein consisting of the amino acid sequence, wherein one or more amino acid residues are deleted, substituted or added in the amino acid sequence shown in SEQ ID NO: 13, 15, 17 or 19, and having the activity to improve the growth of the high concentration oxygen-requiring microorganism under low oxygen concentration; and

[6] a protein consisting of the amino acid sequence which has 80% or more homology or identity to the amino acid sequence shown in SEQ ID NO: 13, 15, 17 or 19, and having the activity to improve the growth of the high concentration oxygen-requiring microorganism under low oxygen concentration.

The proteins described in [1] to [6] can be expressed alone or in a combination of two or more to improve the growth under low oxygen concentration. For example, regarding the proteins described in [1] to [3], the proteins comprising the amino acid sequence shown in SEQ ID NOs: 1, 3, 5, 7, 9 and the variants thereof (proteins in which one or more amino acid residues are deleted, substituted or added, or proteins having 80% or more homology or identity) can be expressed alone or in a combination of two or more. Similarly, regarding the proteins described in [4] to [6], the proteins consisting of the amino acid sequence shown in SEQ ID NOs: 13, 15, 17, 19 and the variants thereof can be expressed alone or in a combination of two or more (e.g., three or more, or four) to improve the growth under low oxygen concentration.

The DNAs described in [1] to [6] can be expressed alone or in a combination of two or more to improve the growth under low oxygen concentration. For example, regarding the DNAs described in [1] to [3], DNAs comprising the nucleotide sequence shown in SEQ ID NOs: 2, 4, 6, 8, 10 and DNAs which hybridize with such DNAs under stringent conditions and codes for a protein having an activity to improve the growth under low oxygen concentration can be expressed alone; and the DNAs encoding protein can be expressed in a combination of two or more (e.g., three or more, or four).

The protein consisting of an amino acid sequence wherein one or more amino acid residues are deleted, substituted or added, and having an activity to improve the growth under low oxygen concentration of high concentration oxygen-requiring microorganisms, for example, coryneform bacteria, can be obtained, for example, by introducing a site-directed mutation into a DNA encoding a protein comprising the amino acid sequence shown in SEQ ID NO: 1, 3, 5, 7, 9, 11, 13, 15, 17 or 19 (for example, a DNA comprising the nucleotide sequence shown in SEQ ID NO: 2, 4, 6, 8, 10, 12, 14, 16, 18 or 20) by site-directed mutagenesis described in Molecular Cloning: A Laboratory Manual, Second Edition, Cold Spring Harbor Laboratory Press (1989) (hereinafter simply referred to as “Molecular Cloning, Second Edition”); Current Protocols in Molecular Biology, John Wiley & Sons (1987-1997) (hereinafter simply referred to as “Current Protocols in Molecular Biology”); Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985); etc.

The number of amino acids that are deleted, substituted or added is not particularly limited, and is generally within the range in which deletion, substitution or addition can be done by a known method such as the above site-directed mutagenesis. The number of amino acids is typically 1 to several tens, preferably 1 to 20, more preferably 1 to 10 and still more preferably 1, 2, 3, 4 or 5.

The expression “an amino acid sequence, wherein one or more amino acid residues are added, deleted or substituted in the amino acid sequence shown in SEQ ID NO: 1, 3, 5, 7, 9, 11, 13, 15, 17 or 19” means that the amino acid sequence can include the deletion, substitution or addition of one or a plurality of amino acids at any position in the same sequence.

The position of an amino acid at which one or more amino acids can be deleted or added can be, for example, 1 to several amino acids from the N- or C-terminus of the amino acid sequence shown in SEQ ID NO: 1, 3, 5, 7, 9, 11, 13, 15, 17 or 19.

Deletion, substitution and addition can be simultaneously included in one sequence, and amino acids to be substituted or added can be natural or non-natural. Examples of the natural amino acids include L-alanine, L-asparagine, L-aspartic acid, L-glutamine, L-glutamic acid, glycine, L-histidine, L-isoleucine, L-leucine, L-lysine, L-arginine, L-methionine, L-phenylalanine, L-proline, L-serine, L-threonine, L-tryptophan, L-tyrosine, L-valine, L-cysteine and the like.

Examples of the mutually substitutable amino acids are shown below. The amino acids in the same group are mutually substitutable.

Group A: leucine, isoleucine, norleucine, valine, norvaline, alanine, 2-aminobutanoic acid, methionine, 0-methylserine, t-butylglycine, t-butylalanine and cyclohexylalanine.

Group B: aspartic acid, glutamic acid, isoaspartic acid, isoglutamic acid, 2-aminoadipic acid and 2-aminosuberic acid.

Group C: asparagine and glutamine.

Group D: lysine, arginine, ornithine, 2,4-diaminobutanoic acid and 2,3-diaminopropionic acid.

Group E: proline, 3-hydroxyproline and 4-hydroxyproline.

Group F: serine, threonine and homoserine.

Group G: phenylalanine and tyrosine.

In the production process of the present invention, to impart the activity of improving the growth of the high concentration oxygen microorganism under low oxygen concentration to a protein, it is desirable that the protein of the present invention has 80% or more, preferably 90% or more, more preferably 95% or more, still more preferably 98% or more and particularly preferably 99% or more homology or identity to the amino acid sequence shown in SEQ ID NO: 1, 3, 5, 7, 9, 11, 13, 15, 17 or 19.

The homology or identity among amino acid sequences or nucleotide sequences can be determined by using algorithm BLAST by Karlin and Altschul (Pro. Natl. Acad. Sci. USA, 90, 5873 (1993)) and FASTA (Methods Enzymol., 183, 63 (1990)). Programs called BLASTN and BLASTX have been developed based on the algorithm BLAST (J. Mol. Biol. 215: 403 (1990)). For BLASTN analysis of a nucleotide sequence based on BLAST, the parameters are set, for example, to score=100, and word length=12. For BLASTX analysis of an amino acid sequence based on BLAST, the parameters are set, for example, to score=50, and word length=3. To obtain gapped alignments, Gapped BLAST can be utilized as described in Altschul et al. (1997, Nucleic Acids Res. 25: 3389-3402). Alternatively, PSI-Blast or PHI-Blast can be used to perform an iterated search that detects distant relationships between molecules (Id.), and relationships between molecules that share a common pattern. When utilizing BLAST, Gapped BLAST, PSI-Blast and PHI-Blast programs, the default parameters of the respective programs can be used. See http://www.ncbi.nlm.nih.gov.

Whether the protein has an activity of improving the growth of a microorganism under low oxygen concentration can be confirmed by checking whether the growth speed under low oxygen concentration of high concentration oxygen-requiring coryneform bacteria in which the protein is expressed is restored to 80% or more, preferably 90% or more compared with that of a wild-type strain.

Whether the proteins described in [4] to [6] have an activity to improve the growth under low oxygen concentration can be confirmed when the ribonucleotide reductase activity of the microorganism transformed with the DNAs encoding the proteins described in [4] to [6] is compared with that of a wild-type strain according to the method described in Microbiology, 145, 1595 (1999), and the microorganism has an improved ribonucleotide reductase activity compared to that of the wild-type strain.

Examples of the DNA that transforms the microorganism used in the production process of the present invention are DNAs following [7] to [12]:

[7] DNA encoding the protein consisting of the amino acid sequence shown in SEQ ID NO: 1, 3, 5, 7, 9 or 11;

[8] DNA comprising the nucleotide sequence shown in SEQ ID NO: 2, 4, 6, 8, 10 or 12;

[9] DNA which hybridizes with a DNA consisting of the nucleotide sequence complementary to the nucleotide sequence shown in SEQ ID NO: 2, 4, 6, 8, 10 or 12 under stringent conditions, and codes for the protein having the activity to improve the growth of the high concentration oxygen-requiring microorganism under low oxygen concentration;

[10] DNA encoding the protein consisting of the amino acid sequence shown in SEQ ID NO: 13, 15, 17 or 19;

[11] DNA consisting of the nucleotide sequence shown in SEQ ID NO: 14, 16, 18 or 20; and

[12] DNA which hybridizes with DNA consisting of the nucleotide sequence complementary to the nucleotide sequence shown in SEQ ID NO: 14, 16, 18 or 20 under stringent conditions, and encodes for the protein having the activity to improve the growth of a high concentration oxygen-requiring microorganism under low oxygen concentration.

The term “hybridize” in this specification indicates that a DNA hybridizes under specific conditions with another DNA having a specific nucleotide sequence or a portion of the DNA. Thus, the nucleotide sequence of the DNA having a specific nucleotide sequence or portion of the DNA can have a length such that the DNA or portion thereof can be used as a probe in Northern or Southern blot analysis or as oligonucleotide primers in PCR analysis. Examples of the DNA used as a probe include DNAs that are 100 nucleotides or more, preferably 200 nucleotides or more and more preferably 500 nucleotides or more in length. DNAs of 10 nucleotides or more, and preferably 15 nucleotides or more in length can also be used.

Methods for DNA hybridization experiments are well known. Such experiments can be conducted by determining hybridization conditions, for example, according to the disclosures in Molecular Cloning, Second and Third Editions (2001); Methods for General and Molecular Bacteriology, ASM Press (1994); Immunology Methods Manual, Academic Press (Molecular); and many other standard textbooks.

Hybridization under stringent conditions as mentioned above can be carried out, for example, in the following manner. A filter with DNA immobilized thereon and a probe DNA are incubated in a solution containing 50% formamide, 5×SSC (750 mM sodium chloride and 75 mM sodium citrate), 50 mM sodium phosphate (pH 7.6), 5×Denhardt's solution, 10% dextran sulfate and 20 μg/l denatured salmon sperm DNA at 42° C. overnight, followed by washing the filter in 0.2×SSC solution of about 65° C. Less-stringent conditions can also be employed. The stringent conditions can be modified by adjusting the concentration of formamide (the conditions become less stringent as the concentration of formamide is lowered) and by changing the salt concentration and the temperature conditions. Hybridization under less-stringent conditions can be carried out, for example, by incubating a filter with DNA immobilized thereon and a probe DNA in a solution containing 6×SSCE (20×SSCE: 3 mol/l sodium chloride, 0.2 mo1/1 sodium dihydrogenphosphate and 0.02 mol/l EDTA, pH 7.4), 0.5% SDS, 30% formamide and 100 μg/l denatured salmon sperm DNA at 37° C. overnight, and washing the filter with 1×SSC solution containing 0.1% SDS of 50° C. Hybridization under still less-stringent conditions can be carried out, for example, by performing hybridization using a solution having a high salt concentration (for example, 5×SSC) under the above-mentioned less-stringent conditions, followed by washing.

The various conditions described above can also be set by adding or changing a blocking reagent to suppress the background in hybridization experiments. The addition of a blocking reagent as described above can also be accompanied by the alteration of hybridization conditions to make the conditions suitable for the purpose.

Examples of the DNA that hybridizes under stringent conditions include DNA having 80% or more, preferably 90% or more, more preferably 95% or more, still more preferably 98% or more and particularly preferably 99% or more homology or identity to the nucleotide sequence shown in SEQ ID NO: 2, 4, 6, 8, 10, 12, 14, 16, 18 or 20 as calculated by the use of programs such as BLAST or FASTA described above, based on the above parameters.

Whether a DNA that hybridizes with the above-mentioned DNA under stringent conditions is a DNA that encodes a protein having an activity to improve the growth of the microorganism under low oxygen concentration can be confirmed by checking whether the growth speed under low oxygen concentration of the transformant obtained by introducing the DNA into a high concentration oxygen-requiring microorganism, which is used as a host cell, is 80% or more, and preferably 90% or more compared with that of the wild-type strain.

The DNA that transforms the microorganism used in the production process of the present invention can be obtained from a microorganism belonging to coryneform bacteria by PCR using chromosomal DNA prepared by the method of Saito et al. (Biochim. Biophys. Acta, 72, 619 (1963)) as a template and using primer DNA designed and synthesized based on the nucleotide sequence shown in SEQ ID NO: 2, 4, 6, 8, 10, 12, 14, 16, 18 or 20.

The DNA of the present invention can be obtained, for example, by PCR using chromosomal DNA prepared from Corynebacterium glutamicum ATCC13032 or ATCC31833 as a template, and using DNA having 5′-terminal and 3′-terminal regions of the nucleotide sequence shown in SEQ ID NO: 2, 4, 6, 8, 10, 12, 14, 16, 18 or 20 as a primer set.

Specific examples of the DNA of the present invention that can be obtained include DNA comprising the nucleotide sequence shown in SEQ ID NO: 2, 4, 6, 8, 10, 12, 14, 16, 18 or 20.

The nucleotide sequences shown in SEQ ID NOs: 2, 4, 6, 8, 10, 12, 14, 16, 18 and 20 are disclosed as Cgl0807, Cgl0600, Cgl1427, Cgl1102, Cgl2859, Cgl2857, Cgl2525, Cgl2530, Cgl2531 and Cgl2532, respectively, in the nucleotide sequence represented by NCBI GenBank accession No. BA000036.

Cgl0807, Cgl0600, Cgl1427, Cgl1102, Cgl2859, Cgl2857, Cgl2525, Cgl2530, Cgl2531 and Cgl2532 respectively correspond to NCgl0773, NCgl0575, NCgl1372, NCgl1057, NCgl2761, NCgl2760, NCgl2438, NCgl2443, NCgl2444 and NCgl2445 in the nucleotide sequence shown in GenBank accession No. NC_{—}003450.

The DNA of the present invention can also be obtained by a hybridization method using partial or full-length DNA consisting of the nucleotide sequence shown in SEQ ID NO: 2, 4, 6, 8, 10, 12, 14, 16, 18 or 20 as a probe; or by a known method such as chemical synthesis based on the nucleotide sequence shown in SEQ ID NO: 2, 4, 6, 8, 10, 12, 14, 16, 18 or 20.

The DNA of the present invention or DNA used in the production process of the present invention can also be obtained by conducting a search through various gene sequence databases for a sequence having 85% or more, preferably 90% or more, more preferably 95% or more, still more preferably 98% or more and particularly preferably 99% or more homology or identity to a nucleotide sequence of DNA encoding the amino acid sequence shown in SEQ ID NO: 1, 3, 5, 7, 9, 11, 13, 15, 17, or 19; and obtaining the desired DNA based on the nucleotide sequence obtained by the search from a chromosomal DNA or cDNA library of an organism having the nucleotide sequence according to the above-described method.

The nucleotide sequence of the DNA can be determined by a conventional sequencing method such as the dideoxy method (Proc. Natl. Acad. Sci., USA, 74, 5463 (1977)), or by using a nucleotide sequencer such as a 373A DNA Sequencer (a product of Perkin-Elmer Corp.).

In cases where the obtained DNA is found to be a partial DNA by the nucleotide sequence analysis, the full-length DNA can be obtained by Southern hybridization for a chromosomal DNA library using the partial DNA as a probe.

The obtained DNA can be used as-is. If necessary, the DNA can be modified by substituting one or more nucleotides to obtain the codon that is most suitable for the expression of coryneform bacteria. Further, if necessary, a DNA fragment having a suitable length and containing a region that encodes the protein of the present invention can be prepared. The DNA or DNA fragment is ligated downstream of a promoter of an expression vector suitable for the expression of coryneform bacteria to produce a recombinant DNA.

The vectors that can be preferably used are those capable of autonomous replication or integration into the chromosome in a microorganism and containing a promoter at a position appropriate for the transcription of DNA to be transformed.

When a prokaryotic organism such as bacteria is used as a host cell, it is preferable that the recombinant DNA having the DNA of the present invention is capable of autonomous replication in a prokaryotic organism and comprise a promoter, a ribosome binding sequence, the DNA of the present invention and a transcription termination sequence. A gene that controls a promoter can also be contained in the recombinant DNA.

When coryneform bacteria is used as a host cell, pCG1 (Japanese Unexamined Patent Publication No. S57-134500); pCG2 (Japanese Unexamined Patent Publication No. S58-35197); pCG4 (Japanese Unexamined Patent Publication No. S57-183799); pCG11 (Japanese Unexamined Patent Publication No. S57-134500); pCG116, pCE54 and pCB101 (all disclosed in Japanese Unexamined Patent Publication No. S58-105999); pCE51, pCE52 and pCE53 (all described in Molecular and General Genetics, 196, 175 (1984)); and the like are preferably used.

The recombinant DNA produced by inserting the DNA of the present invention into a vector preferably comprises a promoter, a ribosome binding sequence, the DNA of the present invention and a transcription termination sequence. A gene that controls a promoter can also be contained in the recombinant DNA.

Any promoter that functions in host cells (coryneform bacteria) can be used. Examples of promoters include promoters derived from E. coli or phages such as trp promoter (Pₜᵣₚ), lac promoter, P_{L} promoter, P_{R} promoter and T7 promoter. Artificially designed or modified promoters can also be used, and examples thereof include a promoter wherein two Pₜᵣₚ are tandemly arranged (Pₜᵣₚ×2), tac promoter, T7-lac promoter, let I promoter and P54-6 promoter for expression in microorganisms belonging to the genus Corynebacterium (Appl. Microbiol. Biotechnol., 53, 674-679 (2000)).

It is preferable that the distance between the Shine-Dalgarno sequence, which is a ribosome-binding sequence, and the initiation codon is adjusted appropriately (for example, 6 to 18 nucleotides). Although a transcription termination sequence is not always necessary, it is preferable to place a transcription termination sequence immediately downstream of the structural gene.

As a method for introducing the recombinant DNA, any method capable of introducing DNA into bacteria can be used. Examples of the methods include the method using calcium ions (Proc. Natl. Acad. Sci., USA, 69, 2110 (1972)), the protoplast method (Japanese Unexamined Patent Publication No. 1988-248394), the electroporation method (Nucleic Acids, Res., 16, 6127 (1988)) and the like.

The DNA encoding a protein having an activity to improve the growth of high concentration oxygen-requiring microorganism under low oxygen concentration can be integrated into a chromosome of coryneform bacteria, or can be introduced as a plasmid into coryneform bacteria to form a transformant. The DNA that has an activity to improve the growth of high concentration oxygen-requiring microorganism under low oxygen concentration is preferably derived from coryneform bacteria.

When a microorganism used as a host cell has an ability to produce a useful substance such as an amino acid, a nucleic acid, etc., the obtained transformant can be used in the production of the useful substance without any treatment. However, when a microorganism does not have such an ability, a useful substance can be obtained by breeding a wild-type strain according to known methods.

Examples of the known methods include the following:

(a) the method of reducing or removing at least one mechanism of controlling the biosynthesis of a useful substance;
(b) the method of enhancing the expression of at least one enzyme involved in the biosynthesis of a useful substance;
(c) the method of increasing the copy number of at least one enzyme gene involved in the biosynthesis of a useful substance;
(d) the method of attenuating or blocking at least one metabolic pathway to produce metabolites other than a useful substance, which branches off from a biosynthetic pathway of the useful substance; and
(e) the method of selecting a cell strain that is highly resistant to an analogue of a useful substance, as compared with a wild-type strain.

The above known methods can be used alone, or in a combination of two or more.

Specific examples of the above methods (a) to (e), for example, to produce an amino acid as a useful substance are described in the following publications. Method (a) is described in Agric. Biol. Chem., 43, 105-111 (1979); J. Bacteriol., 110, 761-763 (1972); Appl. Microbiol. Biotechnol., 39, 318-323 (1993); and the like. Method (b) is described in Agric. Biol. Chem., 43, 105-111 (1979); J. Bacteriol., 110, 761-763 (1972); and the like. Method (c) is described in Appl. Microbiol. Biotechnol., 39, 318- 323 (1993); Agric. Biol. Chem., 39, 371-377 (1987); and the like. Method (d) is described in Appl. Environ. Microbiol., 38, 181-190 (1979); Agric. Biol. Chem., 42, 1773-1778 (1978); and the like. Method (e) is described in Agric. Biol. Chem., 36, 1675-1684 (1972); Agric. Biol. Chem., 41, 109-116 (1977); Agric. Biol. Chem., 37, 2013-2023 (1973); Agric. Biol. Chem., 51, 2089-2094 (1987); and the like. As reference to these publications, microorganisms capable of forming and accumulating various amino acids can be bred and obtained.

Further, many methods for breeding a microorganism capable of forming an amino acid according to any one of, or a combination of the above methods (a) to (e) are described in Biotechnology 2nd ed., vol. 6, Products of Primary Metabolism (VCH Verlagsgesellschaft mbH, Weinheim, 1996), section 14a or 14b; Advances in Biochemical Engineering/Biotechnology 79, 1-35 (2003); and Amino san Hakko (Amino acid fermentation), Japan Scientific Societies Press, Hiroshi Aida et al. (1986). Specific methods for breeding a microorganism capable of forming and accumulating an amino acid are also described in many reports other than the above-mentioned publications, such as Japanese Unexamined Patent Publication No. 2003-164297; Agric. Biol. Chem., 39, 153-160 (1975); Agric. Biol. Chem., 39, 1149-1153 (1975); Japanese Unexamined Patent Publication No. 1983-13599; J. Gen. Appl. Microbiol., 4, 272-283, (1958); Japanese Unexamined Patent Publication No. 1988-94985; Agric. Biol. Chem., 37, 2013-2023 (1973); WO 97/15673; Japanese Unexamined Patent Publication No. 1981-18596; Japanese Unexamined Patent Publication No. 1981-144092; and Japanese Unexamined Patent Publication No. 2003-511086. As reference to these publications, a microorganism having an ability to produce an amino acid can be bred.

The useful substance can be obtained by collecting the useful substance produced and accumulated in a culture obtained by culturing a transformant in a medium using the production process of the present invention.

The transformant used in the production process of the present invention is cultured according to an ordinal method for culturing a microorganism. It is preferable to use the most appropriate conditions depending on a useful substance to be produced.

The carbon sources, nitrogen sources, inorganic salts and other additives of the medium for culturing the transformant are the same as those used in the medium for determining the high concentration oxygen requirement.

Culturing of the transformant is performed under aerobic conditions by a shaking culture, deep aeration agitation culture or the like. In general, the culture temperature is from 20 to 42° C. and preferably from 30 to 40° C. The pH of the medium is preferably maintained in the range of pH 5 to pH 9. The pH of the medium is adjusted by using an inorganic or organic acid, alkaline solution, urea, calcium carbonate, ammonia, etc.

The culturing period generally ranges from 1 to 6 days. During cultivation, antibiotics such as ampicillin and tetracycline may be added to the medium, if necessary.

When the microorganism transformed with a recombinant DNA using an inductive promoter as a promoter is cultured, an inducer may be added to the medium, if necessary. For example, when the microorganism transformed with the recombinant vector comprising lac promoter is cultured, isopropyl-β-D-thiogalactopyranoside, etc. may be added to the medium; when the microorganism transformed with the recombinant vector comprising a trp promoter is cultured, indole acrylic acid etc. may be added to the medium.

Regarding the useful substance produced and accumulated in the culture, precipitates such as cells are removed from the culture, and then the target useful substance can be collected from the culture by using a combination of known methods such as activated carbon treatment and ion exchange resin treatment.

### EXAMPLES

The examples below serve to illustrate the present invention, but should not be construed as limitations on the scope of the invention.

### Example 1

(1) The Corynebacterium glutamicum strain ATCC31833 (hereinafter referred to as ATCC31833 strain) was mutagenized with nitrosoguanidine according to a usual method.

The ATCC31833 strain treated with the mutagen was spread on BY agar medium (20 g of normal bouillon, 5 g of yeast extract and 12 g of Ina agar S-7 in 1 L of water; and adjusted to pH 7.2 by an aqueous sodium hydroxide solution), and then cultured under atmospheric condition (oxygen concentration: 21%) and 0.5% oxygen concentration. The oxygen concentration was set using an Anaero Pack Kenki (produced by Mitsubishi Gas Chemical Co., Inc.), according to the manual therefor. Using the replica method, strains that did not grow well under 0.5% oxygen concentration compared to the strains under atmospheric condition, or strains that could not grow under 0.5% oxygen concentration were selected as high concentration oxygen-requiring strains. From the selected strains, six kinds of variants were selected as representative strains, and they were named as OX-3, OX-96, OX-109, OX-112, OX-119 and OX-150, respectively. The strains were cultured on BY agar medium for 2 days at 30° C. under the various oxygen concentrations shown in Table 1. Table 1 shows the results of the growth of six strains under different oxygen concentrations. In Table 1, “++” indicates good growth, “+” indicates bad growth, and “−” indicates no growth.

TABLE 1
Growth under each oxygen concentration

| | Oxygen concentration | | | | |
|---|---|---|---|---|---|
| Strains | 21% | 9% | 6% | 3% | 0.5% |
| ATCC31833 | ++ | ++ | ++ | ++ | + |
| OX-3 | ++ | + | − | − | − |
| OX-96 | + | + | − | − | − |
| OX-109 | + | + | − | − | − |
| OX-112 | + | + | − | − | − |
| OX-119 | + | + | − | − | − |
| OX-150 | ++ | ++ | ++ | + | − |

As shown in Table 1, the strains OX-3, OX-96, OX-109, OX-112, OX-119 and OX-150 showed high concentration oxygen-requiring properties as compared to the parent strain ATCC31833.

(2) The chromosomal DNA of ATCC31833 strain was prepared according to the method described in Japanese Unexamined Patent Publication No. H6-169785.

The chromosomal DNA of ATCC31833 strain and plasmid pCSEK20 (W001/021774) were individually digested with EcoRI, and the fragments were ligated with a ligation kit. The chromosomal DNA of ATCC31833 strain and plasmid pCS299P (Appl. Microbiol. Biotech., 63, 592 (2004)) were individually digested with BamHI, and then the fragments were ligated with a ligation kit.

The high concentration oxygen-requiring strains obtained in (1), i.e., OX-3, OX-96, OX-109, OX-112, OX-119 and OX-150 were transformed with the thus-prepared two kinds of genomic libraries, according to the method described in Japanese Unexamined Patent Publication No. H6-169785.

From the obtained transformants, strains that were capable of growing under 0.5% oxygen concentration in BY agar medium containing 20 μg/mL of kanamycin were selected as transformants in which high concentration oxygen-requiring properties were restored. According to the vector preparation method described in Japanese Unexamined Patent Publication No. H6-169785, plasmid DNA was isolated from each of the selected transformants.

Each of the obtained various plasmid DNAs was digested and analyzed with restriction enzymes. As a result, 1.9 Kb EcoRI cleavage fragment was obtained as a DNA fragment for restoring the high concentration oxygen-requiring properties of OX-3, 2.1 Kb BamHI cleavage fragment as a DNA fragment for restoring the high concentration oxygen-requiring properties of OX-96, 3.2 Kb EcoRI cleavage fragment as a DNA fragment for restoring the high concentration oxygen-requiring properties of OX-109, 1.1 Kb EcoRI cleavage fragment as a DNA fragment for restoring the high concentration oxygen-requiring properties of OX-112, 1.8 Kb BamHI cleavage fragment and 3.2 Kb BamHI cleavage fragment as DNA fragments for restoring the high concentration oxygen-requiring properties of OX-119, and 4.4 Kb EcoRI cleavage fragment as a DNA fragment for restoring the high concentration oxygen-requiring properties of OX-150. The thus-obtained plasmid containing 1.9 Kb EcoRI fragment, plasmid containing 2.1 Kb BamHI fragment, plasmid containing 3.2 Kb EcoRI fragment, plasmid containing 1.1 Kb EcoRI fragment, plasmid containing 1.8 Kb BamHI fragment, plasmid containing 3.2 Kb BamHI fragment and plasmid containing 4.4 Kb EcoRI fragment were named as pEcol.9, pBam2.1, pEco3.2, pEco1.1, pBam1.8, pBam3.2 and pEco4.4, respectively.

The nucleotide sequence of each DNA fragment inserted in pEco1.9, pBam2.1, pEco3.2, pEco1.1, pBam1.8, pBam3.2 and pEco4.4 was determined according to a known method.

As a result of the analysis of the determined nucleotide sequences based on the entire genome information of the Corynebacterium glutamicum (NCBI GenBank Accession No. BA000036), Cgl0807 was present in the 1.9 Kb EcoRI fragment contained in the pEco1.9. Cgl1101 and Cgl1102 were present in the 2.1 Kb BamHI fragment contained in the pBam2.1. Cgl2859 and Cgl2861 were present in the 3.2 Kb EcoRI fragment contained in the pEco3.2. Cgl0600 was present in the 1.1 Kb EcoRI fragment contained in the pEco1.1. Cgl1427 was present in the 1.8 Kb BamHI fragment contained in the pBam1.8. Cgl2857 and Cgl2858 were present in the 3.2 Kb BamHI fragment contained in the pBam3.2. Four genes, i.e., Cgl2530, Cgl2531, Cgl2532 and Cgl2533, were present in the 4.4 Kb EcoRI fragment contained in pEco4.4.

The amino acid sequence inferred from the nucleotide sequence of the Cgl0807 gene that complements the high concentration oxygen-requiring properties of OX-3 strain is shown in SEQ ID NO: 1, and the nucleotide sequence is shown in SEQ ID NO: 2.

The amino acid sequence inferred from the nucleotide sequence of the Cgl0600 gene that satisfies the high concentration oxygen-requiring properties of OX-112 strain is shown in SEQ ID NO: 3, and the nucleotide sequence is shown in SEQ ID NO: 4.

The amino acid sequence inferred from the nucleotide sequence of the Cgl1427 gene that satisfies the high concentration oxygen-requiring properties of OX-119 strain is shown in SEQ ID NO: 5, and the nucleotide sequence is shown in SEQ ID NO: 6.

The ability of restoring the high concentration oxygen-requiring properties of OX-96 strain was examined by subcloning various DNA fragments from pBam2.1 according to a usual method. The results showed that the high concentration oxygen-requiring properties of OX-96 strain were restored by the plasmid pCgl1102 that contains a Cgl1102 gene alone. Plasmid pCgl1102 was obtained by treating a 0.5 Kb DNA fragment and vector pCS299P with BamHI restriction enzyme, wherein the 0.5 Kb of DNA fragment was obtained by PCR using plasmid pBam2.1 as a template and DNAs consisting of the nucleotide sequences shown in SEQ ID NOs: 21 and 22 as primers, and then ligating them to each other. The amino acid sequence inferred from the nucleotide sequence of Cgl1102 gene is shown in SEQ ID NO: 7, and the nucleotide sequence is shown in SEQ ID NO: 8.

Similarly, the ability of restoring the high concentration oxygen-requiring properties of OX-109 strain was also examined by subcloning various DNA fragments from pEco3.2 according to a usual method. As a result, the high concentration oxygen-requiring properties of OX-109 strain was restored by the plasmid pCgl2859 containing a Cgl2859 gene alone. Plasmid pCgl2859 was obtained by digesting with BamHI and removing Cgl2861 from plasmid pEco3.2 and ligating them again. The amino acid sequence inferred from the nucleotide sequence of Cgl2859 gene is shown in SEQ ID NO: 9, and the nucleotide sequence is shown in SEQ ID NO: 10.

The ability of restoring the high concentration oxygen-requiring properties of OX-119 strain was also examined by subcloning various DNA fragments from pBam3.2 according to a usual method. As a result, the high concentration oxygen-requiring OX-119 strain was restored by the plasmid pCgl2857 containing a Cgl2857 gene alone. Plasmid pCgl2857 was obtained by treating a 1.4 Kb DNA fragment and vector pCS299P with BamHI restriction enzyme, wherein the 1.4 Kb DNA fragment was obtained by PCR using plasmid pBam3.2 as a template and DNAs consisting of the nucleotide sequences shown in SEQ ID NOs: 23 and 24 as primers, and then ligating them to each other. The amino acid sequence inferred from the nucleotide sequence of Cgl2857 gene is shown in SEQ ID NO: 11, and the nucleotide sequence is shown in SEQ ID NO: 12.

The ability of restoring the high concentration oxygen-requiring properties of OX-150 strain was also examined by subcloning various DNA fragments from pEco4.4 according to a usual method. As a result, the high concentration oxygen-requiring properties of OX-150 strain were restored by the plasmid pHIE containing genes Cgl2530, Cgl2531 and CGl2532. Plasmid pHIE was obtained by treating a 3.8 Kb DNA fragment and vector pCS299P with KpnI restriction enzyme, wherein the 3.8 Kb DNA fragment was obtained by PCR using chromosomal DNA of ATCC31833 strain as a template and DNAs consisting of the nucleotide sequences shown in SEQ ID NOs: 25 and 26 as primers, and then ligating them to each other.

It is reported that these three genes are ribonucleotide reductase-related proteins, and that Cgl2525 is a constituting factor of the enzyme (Microbiology, 145, and 1595 (1999)). The plasmid pHIEF containing these 4 genes was constructed, and the complementation of the high concentration oxygen-requiring properties of OX-150 strain was examined. As a result, the plasmid pHIEF containing all 4 genes showed higher complementation ability than the plasmid pHIE containing only 3 genes. pHIEF was obtained by treating a 1.6 Kb DNA fragment and vector pCS299P with BamHI restriction enzyme, wherein the 1.6 Kb DNA fragment was obtained by PCR using chromosomal DNA of ATCC31833 strain as a template and DNAs consisting of the nucleotide sequences shown in SEQ ID NOs: 27 and 28 as primers, and then ligating them to each other.

The amino acid sequence inferred from the nucleotide sequence of Cgl2525 gene is shown in SEQ ID NO: 13, and the nucleotide sequence is shown in SEQ ID NO: 14. The amino acid sequence inferred from the nucleotide sequence of Cgl2530 gene is shown in SEQ ID NO: 15, and the nucleotide sequence is shown in SEQ ID NO: 16. The amino acid sequence inferred from the nucleotide sequence of Cgl2531 gene is shown in SEQ ID NO: 17, and the nucleotide sequence is shown in SEQ ID NO: 18. The amino acid sequence inferred from the nucleotide sequence of Cgl2532 gene is shown in SEQ ID NO: 19, and the nucleotide sequence is shown in SEQ ID NO: 20.

(3) The following strains were cultured in BY agar medium for 2 days at 30° C. under the different oxygen concentrations shown in Table 2: OX-3 strain, OX-3/pEco1.9 strain in which plasmid pEco1.9 was introduced in OX-3 strain; OX-96 strain, OX-96/pCgl1102 strain in which plasmid pCgl1102 was introduced in OX-96 strain; OX-109 strain, OX-109/pCgl2859 strain in which plasmid pCgl2859 was introduced in OX-109 strain; OX-112 strain, OX-112/pEco1.1 strain in which plasmid pEco1.1 was introduced in OX-112 strain; OX-119 strain, OX-119/pBam1.8 strain in which plasmid pBam1.8 was introduced in OX-119 strain; OX-119/pCgl2857 strain in which plasmid pCgl2857 was introduced in OX-119 strain; and OX-150 strain, and OX-150/pHIEF strain in which plasmid pHIEF was introduced in OX-150. Table 2 shows the results. In Table 2, “++” indicates good growth, “+” indicates bad growth, and “−” indicates no growth.

TABLE 2
Growth under each oxygen concentration

| | Oxygen concentration | | | | |
|---|---|---|---|---|---|
| Strains | 21% | 9% | 6% | 3% | 0.5% |
| ATCC31833 | ++ | ++ | ++ | ++ | + |
| OX-3 | ++ | + | − | − | − |
| OX-3/pEco1.9 | ++ | ++ | + | + | + |
| OX-96 | + | + | − | − | − |
| OX-96/pCgl1102 | ++ | ++ | + | + | + |
| OX-109 | + | + | − | − | − |
| OX-109/pCgl2859 | ++ | ++ | ++ | + | + |
| OX-112 | + | + | − | − | − |
| OX-112/pEco1.1 | ++ | ++ | + | + | + |
| OX-119 | + | + | − | − | − |
| OX-119/pBam1.8 | ++ | ++ | + | − | − |
| OX-119/pCgl2857 | ++ | ++ | ++ | + | + |
| OX-150 | ++ | ++ | ++ | + | − |
| OX-150/pHIEF | ++ | ++ | ++ | ++ | + |

As shown in Table 2, in the OX-3/pEcol.9 strain, OX-96/pCgl1102 strain, OX-109/pCgl2859 strain, OX-112/pEco1.1 strain, OX-119/pBam1.8 strain, OX-119/pCgl2857 strain and OX-150/pHIEF strain, high concentration oxygen-requiring properties of those strains were restored compared to the each host strains.

### Example 2

An L-lysine- producing bacterium was transformed with plasmid pEco1.9, pCgl1102, pCgl2859, pEco1.1, pBam1.8, pCgl2857 and pHIEF obtained in Example 1, according to the method described in Japanese Unexamined Patent Publication No. H6-169785. As the L-lysine-producing bacterium, a Corynebacterium glutamicum AHP-3 strain (FERN BP-7382), whose genetic character was known, was used. The Corynebacterium glutamicum AHP-3 strain has Va159Ala amino acid substitution in the homoserine dehydrogenase gene (hom), Thr331IIe amino acid substitution in the aspartokinase gene (lysC), and Pro458Ser amino acid substitution in the pyruvate carboxylase gene (pyc) on the chromosome of wild strain ATCC13032 of the Corynebacterium glutamicum. According to the preparation method of a vector disclosed in Japanese Unexamined Patent Publication No. 1994-169785, plasmid DNA was isolated from each of the transformed strains having kanamycin resistance. It was confirmed that each transformed strain had each plasmid by the cleavage analysis of the plasmids using various restriction enzymes.

The L-lysine production tests of these transformed strains and parent strains were conducted as follows. One platinum loop amount of cells which was cultured at 30° C. for 24 hours on the BY agar medium (7 g of meat extract, 10 g of peptone, 3 g of sodium chloride, 5 g of yeast extract, and 15 g of Bacto agar in 1 L of water; and adjusted to pH 7.2), was inoculated into a large test tube containing 5mL of seed medium (20 g of glucose, 7 g of meat extract, 10 g of peptone, 3 g of sodium chloride, and 5 g of yeast extract in 1 L of water; and adjusted to pH 7.2, after which 10 g of calcium carbonate was added), and cultured at 30° C. for 13 hours. 0.5mL of this seed culture solution was inoculated into a large test tube containing 7 mL of main culture medium (50 g of glucose, 10 g of corn steep liquor, 45 g of ammonium sulfate, 2 g of urea, 0.5 g of potassium dihydrogen phosphate, 0.5 g of magnesium sulfate heptahydrate, and 0.3 mg of biotin in 1 L of water; and adjusted to pH 7.0, after which 30 g of calcium carbonate was added), and cultured on a shaker at 30° C. for 72 hours. After the cells were removed from the culture by centrifugation, the amount of L-lysine monohydrochloride accumulated in the supernatant was determined by high-performance liquid chromatography (HPLC). Table 3 shows the results.

TABLE 3
Test of L-lysine Production

| | | L-lysine |
|---|---|---|
| | | hydrochloride |
| Strains | Growth (OD₆₆₀) | (g/l) |
| AHP-3 | 15 | 4.4 |
| AHP-3/pEco1.9 | 16 | 5.0 |
| AHP-3/pCgl1102 | 16 | 5.0 |
| AHP-3/pCgl2859 | 17 | 5.2 |
| AHP-3/pEco1.1 | 16 | 5.1 |
| AHP-3/pBam1.8 | 18 | 5.2 |
| AHP-3/pCgl2857 | 17 | 5.2 |
| AHP-3/pHIEF | 15 | 5.0 |

As is clear from Table 3, the AHP-3 strain having the plasmid containing the gene of the present invention produced L-lysine in a significantly increased amount, as compared to the parent strain AHP-3.

### INDUSTRIAL APPLICABILITY

According to the present invention, a process for producing a useful substance using a transformant transformed with a DNA encoding a protein having an activity to improve the growth of a high concentration oxygen-requiring microorganism under low oxygen concentration can be provided.

SEQ ID NO: 21 —Description of Artificial Sequence: Synthetic DNA SEQ ID NO: 22 —Description of Artificial Sequence: Synthetic DNA SEQ ID NO: 23 —Description of Artificial Sequence: Synthetic DNA SEQ ID NO: 24 —Description of Artificial Sequence: Synthetic DNA SEQ ID NO: 25 —Description of Artificial Sequence: Synthetic DNA SEQ ID NO: 26 —Description of Artificial Sequence: Synthetic DNA SEQ ID NO: 27 —Description of Artificial Sequence: Synthetic DNA SEQ ID NO: 28 —Description of Artificial Sequence: Synthetic DNA

1. A process for producing a useful substance which comprises culturing a transfonnant obtained by transforming a microorganism capable of producing the useful substance with a DNA encoding a protein in a medium, wherein the protein has an activity to improve growth of a high concentration oxygen-requiring microorganism under low oxygen concentration;
forming and accumulating the useful substance in a culture; and collecting the useful substance from the culture.

2. The process according to claim 1, wherein the protein having the activity to improve the growth of the high concentration oxygen-requiring microorganism under low oxygen concentration is a protein defined in any one of [1] to [6] below:
[1] a protein comprising the amino acid sequence shown in SEQ ID NO: 1, 3, 5, 7, 9 or 11;
[2] a protein consisting of the amino acid sequence, wherein one or more amino acid residues are deleted, substituted or added in the amino acid sequence shown in SEQ ID NO: 1, 3, 5, 7, 9 or 11, and having the activity to improve the growth of the high concentration oxygen-requiring microorganism under low oxygen concentration;
[3] a protein consisting of the amino acid sequence which has 80% or more homology or identity to the amino acid sequence shown in SEQ ID NO: 1, 3, 5, 7, 9 or 11, and having the activity to improve the growth of the high concentration oxygen-requiring microorganism under low oxygen concentration;
[4] a protein consisting of the amino acid sequence shown in SEQ ID NO: 13, 15, 17 or 19;
[5] a protein consisting of the amino acid sequence, wherein one or more amino acid residues are deleted, substituted or added in the amino acid sequence shown in SEQ ID NO: 13, 15, 17 or 19, and having the activity to improve the growth of the high concentration oxygen-requiring microorganism under low oxygen concentration; and
[6] a protein consisting of the amino acid sequence which has 80% or more homology or identity to the amino acid sequence shown in SEQ ID NO: 13, 15, 17 or 19, and having the activity to improve the growth of the high concentration oxygen-requiring microorganism under low oxygen concentration.

3. The process according to claim 1, wherein the DNA encoding the protein having the activity to improve the growth of the high concentration oxygen-requiring microorganism under low oxygen concentration is a DNA defined in any one of [1] to [6] below:
[1] DNA encoding the protein consisting of the amino acid sequence shown in SEQ ID NO: 1, 3, 5, 7, 9 or 11;
[2] DNA comprising the nucleotide sequence shown in SEQ ID NO: 2, 4, 6, 8, 10 or 12;
[3] DNA which hybridizes with a DNA consisting of the nucleotide sequence complementary to the nucleotide sequence shown in SEQ ID NO: 2, 4, 6, 8, 10 or 12 under stringent conditions, and codes for the protein having the activity to improve the growth of the high concentration oxygen-requiring microorganism under low oxygen concentration;
[4] DNA encoding the protein consisting of the amino acid sequence shown in SEQ ID NO: 13, 15, 17 or 19;
[5] DNA consisting of the nucleotide sequence shown in SEQ ID NO: 14, 16, 18 or 20; and
[6] DNA which hybridizes with DNA consisting of the nucleotide sequence complementary to the nucleotide sequence shown in SEQ ID NO: 14, 16, 18 or 20 under stringent conditions, and encodes for the protein having the activity to improve the growth of the high concentration oxygen-requiring microorganism under low oxygen concentration.

4. The process according to claim 2, wherein the DNA encoding the protein having the activity to improve the growth of the high concentration oxygen-requiring microorganism under low oxygen concentration is a DNA defined in any one of [1] to [6] below:
[1] DNA encoding the protein consisting of the amino acid sequence shown in SEQ ID NO: 1, 3, 5, 7, 9 or 11;
[2] DNA comprising the nucleotide sequence shown in SEQ ID NO: 2, 4, 6, 8, 10 or 12;
[3] DNA which hybridizes with a DNA consisting of the nucleotide sequence complementary to the nucleotide sequence shown in SEQ ID NO: 2, 4, 6, 8, 10 or 12 under stringent conditions, and codes for the protein having the activity to improve the growth of the high concentration oxygen-requiring microorganism under low oxygen concentration;
[4] DNA encoding the protein consisting of the amino acid sequence shown in SEQ ID NO: 13, 15, 17 or 19;
[5] DNA consisting of the nucleotide sequence shown in SEQ ID NO: 14, 16, 18 or 20; and
[6] DNA which hybridizes with DNA consisting of the nucleotide sequence complementary to the nucleotide sequence shown in SEQ ID NO: 14, 16, 18 or 20 under stringent conditions, and encodes for the protein having the activity to improve the growth of the high concentration oxygen-requiring microorganism under low oxygen concentration.
